(19) United States
(12) Patent Application Publication
(10) Pub. No.: US 2005/0124928 A1
(43) Pub. Date: Jun. 9, 2005

(54) Intravascular catheter

(76) Inventor: Robert C. Beck, St. Paul, MN (US)

Correspondence Address: BECK AND TYSVER, 2900 THOMAS AVENUE SOUTH, SUITE 100, MINNEAPOLIS, MN, 55416, US

(21) Appl. No.: 10/950,941

(22) Filed: Sep. 27, 2004

Related U.S. Application Data
(63) Continuation-in-part of application No. 09/637,529, filed on Aug. 11, 2000.
(60) Provisional application No. 60/506,392, filed on Sep. 27, 2003.

(51) Int. Cl. A61B 17/20

(52) U.S. Cl. 604/43

(57) ABSTRACT

A thrombectomy catheter having a secondary cross-flow of fluid driven by a Coanda nozzle shrouded in the distal tip of the device.

This application claims the benefit of U.S. Provisional Application 60/506,392, filed Sep. 27, 2003, and is a continuation in part of Ser. No. 09/637,529 filed Aug. 11, 2000 which is incorporated in its entirety by reference herein.

### FIELD OF THE INVENTION

The present invention relates generally to the field of intravascular catheters, and more particularly to devices that both inject and aspirate fluid from a body lumen.

### BACKGROUND OF THE INVENTION

Catheters that both inject and aspirate are well known in the art and a currently available device of this type is manufactured POSSIS Medical of Minneapolis, Minn., as their Angiojet XMI catheter.

It is important to make the distal tip of such catheters as flexible as possible and current technology, which relies on metal hypodermic tubing, is problematic in this regard. Due to the asymmetrical design of the product, pressures supplied to the nozzle are asymmetric and the hydraulic jet directions vary if an effort is made at reducing the stiffness of the distal tip.

### SUMMARY OF THE INVENTION

In the present invention a Coanda nozzle is used to drive a secondary flow in a catheter sheath having one or more holes. The Coanda nozzle is entirely radially symmetric and operates over a wide pressure range. Distortion due to pressure changes do not adversely effect the operation of the Coanda nozzle.

### BRIEF DESCRIPTION OF THE DRAWINGS

FIG. 1 is a cross-section of the distal tip of a catheter employing the technology.

FIG. 2 is an alternate design of the distal tip of the catheter.

FIG. 3 is an alternate design of the distal tip of the catheter.

FIG. 4 is a digram illustrating the Coanda effect as applied to the FIG. 2 and FIG. 3 embodiment.

### DETAILED DESCRIPTION OF THE DRAWINGS

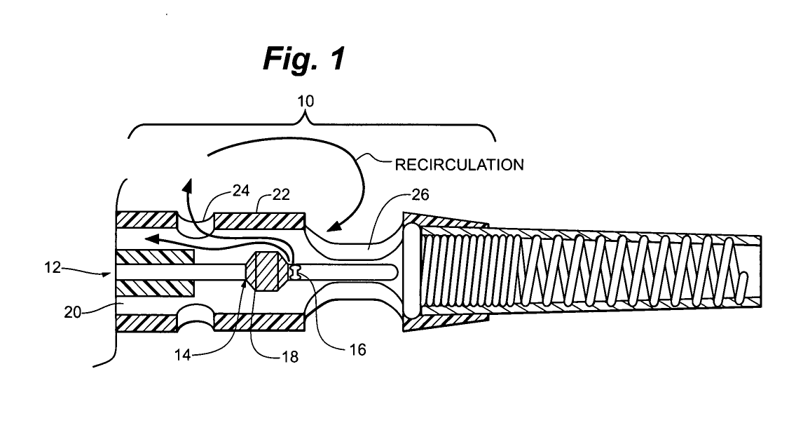

FIG. 1 shows the distal tip 10 of a thrombectomy catheter of the Angiojet XMI type. A fluid inlet 12 lumen couples the hypo tube 14 to a high-pressure injector not shown but also of the Angiojet type. The injected inlet fluid may be saline and it is ejected from holes typified by hole 16. The nubbin 18 is located on the hypo tube and it has a conical surface adjacent the holes. This nubbin forms a wall and the Coanda effect causes fluid to adhere to the wall and flow retrograde out the outlet 20. A portion of this flow exits the catheter body 22 through one or more recirculation holes 24. This flow is re-circulated to the inlet port 26.

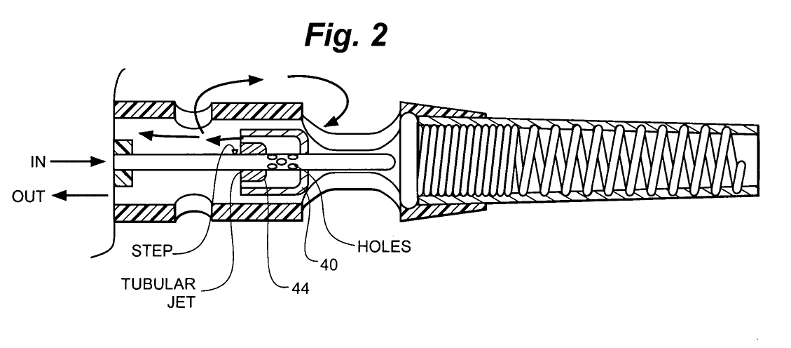

FIG. 2 shows the distal tip 10 of a catheter as well. In this embodiment the hypo tube inlet lumen has a series of holes that communicate to the interior of a cuff 40 that surrounds the hypo tube and is concentric with the hypo tube. A band 44 forms a step at the outlet of the cuff. Together the band and the cuff form an orifice to allow a tubular stream to emerge from the cuff in the retrograde direction although the antegrade direction may be selected as an alternative. The jet of fluid that emerges from the slit formed by the cuff and band flows retrograde. This stream may divide and recirculation as seen in FIG. 1.

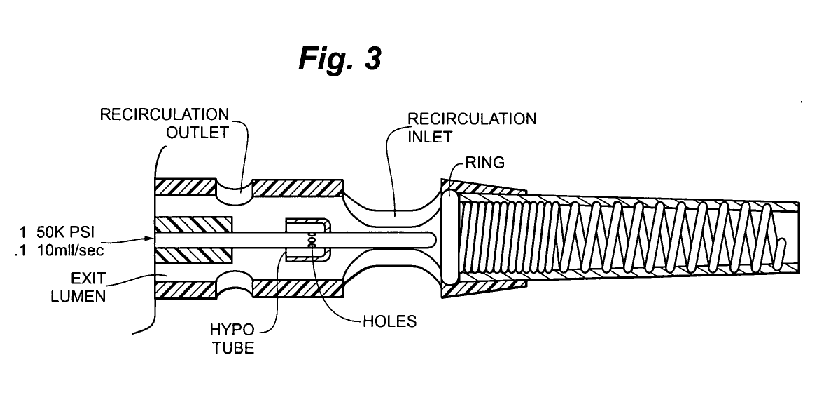

FIG. 3 shows an embodiment that is identical to the FIG. 2 embodiment but it lacks the band 44 so the step seen in FIG. 2 is lacking. It is expected that the tubular jet that emerges from this structure will also “hug” the hypo tube due to the Coanda effect.

In the FIG. 2 and FIG. 3 device the jet as it emerges from the hypo tube through the holes is redirected r retrograde with the cuff. The step in the FIG. 2 embodiment causes the jet to deflect toward the hypo tube. In FIG. 1 the fluid that emerges from the holes is turned by the low-pressure zone on the conical surface of the nubbin 18. In the FIG. 1 embodiment both the leading edge and the trailing edge of the nubbin 18 have conical surfaces.

In FIG. 1 FIG. 2 and FIG. 3 the jet that flows in the catheter body is “tubular” and concentric with the hypo tube and the catheter body.

Coanda Effect

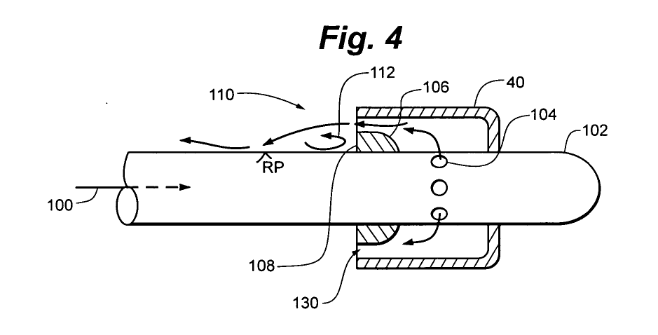

An understanding of the scope of the invention is facilitated by a brief discussion of the Coanda effect as applied to the nozzle shown in FIG. 4. Fluid under pressure 100 is introduced into the tube 102 where it emerges from a series of holes typified by hole 104 near the distal tip of the device. Fluid exiting the hole enters a reservoir formed by cuff 40, which cooperates with a nubbin 106, which together form a step illustrated at numeral 108. The annular flow of fluid exiting from the cuff over the annular step 108 entrains fluid on both the exterior side of the jet identified by arrow 110 and the interior of the jet indicated by recirculation arrow 112. The entrainment and recirculation near the step region causes the jet which emerges from the annular nozzle 130 to attach or adhere to the body of the catheter and in fact strikes the body at a location called the recirculation point or RP in the figure. Only one half of the jet flow is shown for clarity and to provide room for the numerals. This adherence of the emerging jet to the catheter results in a dramatic whirl of turbulence, which is not illustrated in the figure for simplicity. The location of RP has an impact on the performance of the device and RP can be moved closer to the annular slit 130 by reducing the height of the step to a near zero step height. Increasing the step height moves RP in a proximal direction along the length of the catheter. The step height should be non-zero to provide reliable attachment and step heights which correspond roughly to the linear dimension to the annular nozzle 130 are effective at causing wall attachment of the emerging jet to the catheter. This effect occurs with a substantial amount of hysterics and that means that if the nozzle dimensions are deflected due to pressure or mechanical manipulation of the distal tip of the catheter the flow remains reliably attached to the shaft, which is a benefit.

1. A catheter having a proximal and a distal tip:
said distal tip having a first distal hole and a second proximal hole separated by a distance;
a Coanda nozzle located between said proximal hole and distal hole fed by a fluid supply lumen and generating a fluid flow;
whereby fluid is recirculated between said distal aperture and said proximal aperture.
